(19) United States
(12) Patent Application Publication
(10) Pub. No.: US 2017/0292859 A1
(43) Pub. Date: Oct. 12, 2017

(54) Rotary Sensor Component and Method of Manufacture

(71) Applicant: Novo Nordisk A/S, Bagsvaerd (DK)

(72) Inventor: John Oestergaard Madsen, Roedovre (DK)

(21) Appl. No.: 15/517,030

(22) PCT Filed: Oct. 7, 2015
(86) PCT No.: PCT/EP2015/073157

§ 371 Date: Apr. 5, 2017

(30) Foreign Application Priority Data
Oct. 8, 2014 (EP) 14188039.3

(51) Int. Cl. G01D 5/252; A61M 5/315

(52) U.S. Cl. CPC G01D 5/252; A61M 5/31568; A61M 2207/00

(57) ABSTRACT

A method of manufacturing a sensor member comprising the steps of arranging an initial sensor member in a fixed position relative to a material-removing tool, and control the tool to (i) remove material from the carrier alignment portion to thereby create an alignment structure, and (ii) remove material from at least one sensor layer area to thereby create a code segment pattern, each code segment thereby being aligned with the alignment structure.

The present invention relates to components, methods, devices and systems adapted for capturing information in respect of rotational movement. In a specific aspect the invention addresses issues relating to electronic dose data capturing in and for a drug delivery device.

### BACKGROUND OF THE INVENTION

In the disclosure of the present invention reference is mostly made to the treatment of diabetes using a drug delivery device, however, this is only an exemplary use of the present invention.

Drug injection devices have greatly improved the lives of patients who must self-administer drugs and biological agents. Drug injection devices may take many forms, including simple disposable devices that are little more than an ampoule with an injection means or they may be durable devices adapted to be used with pre-filled cartridges. Regardless of their form and type, they have proven to be great aids in assisting patients to self-administer injectable drugs and biological agents. They also greatly assist care givers in administering injectable medicines to those incapable of performing self-injections.

Performing the necessary drug injection at the right time and in the right size is essential for managing diabetes, i.e. compliance with the specified drug regimen is important. In order to make it possible for medical personnel to determine the effectiveness of a prescribed dosage pattern, diabetes patients are encouraged to keep a log of the size and time of each injection. However, such logs are normally kept in handwritten notebooks, from the logged information may not be easily uploaded to a computer for data processing. Furthermore, as only events, which are noted by the patient, are logged, the note book system requires that the patient remembers to log each injection, if the logged information is to have any value in the treatment of the patient's disease. A missing or erroneous record in the log results in a misleading picture of the injection history and thus a misleading basis for the medical personnel's decision making with respect to future medication. Accordingly, it may be desirable to automate the logging of ejection information from medication delivery systems.

Correspondingly, a number of injection devices with a dose monitoring/acquisition feature has been provided, see e.g. in US 2009/0318865, WO 2010/052275 and U.S. Pat. No. 7,008,399. However, most devices of today are without it.

Having regard to the above, it is an object of the present invention to provide a drug delivery device as well as components, assemblies and methods therefore which in a safe, accurate, user-friendly, cost-effective and reliable way allows detection and storage of dose data related to use of a drug delivery device. It is a further object to provide such components and assemblies which could be used also in other applications having the same types of input.

### DISCLOSURE OF THE INVENTION

In the disclosure of the present invention, embodiments and aspects will be described which will address one or more of the above objects or which will address objects apparent from the below disclosure as well as from the description of exemplary embodiments.

Thus, in a first aspect of the invention a method of manufacturing a sensor member is provided. The method comprises the step of (i) providing a sensor member in an initial state, wherein the sensor member comprises a carrier having a surface and an alignment portion, as well as at least one conducting sensor layer area formed on a portion of the carrier surface. The method comprises the further steps of (ii) providing a material-removing tool adapted to remove material from the carrier alignment portion and the sensor layer, (iii) arranging the sensor member in a fixed position relative to the tool, and (iv) control the tool to remove material from the carrier alignment portion to thereby create an alignment structure, and remove material from at least one sensor layer area to thereby create a code segment pattern, each code segment thereby being aligned with the alignment structure. More than one alignment structure may be created.

By combining the two process activities in one step, the tolerances inherent when aligning the surfaces between the two process activities are eliminated, and only the inherent tolerances in the single process of creating the pattern and alignment structure are present. The tool may for example be an ablation laser.

In an exemplary embodiment the created alignment structure is an edge portion of the carrier. The carrier is in the form of a planar board, and the at least one conducting sensor layer area may be formed on the carrier surface by a plating process to thereby create a traditional printed circuit board (PCB).

In exemplary embodiments at least one conducting sensor layer area is ring-formed, the created code segments being in the form of a plurality of annular segments each spanning a given number of degrees.

The provided carrier may have one or more additional surfaces each comprising at least one conducting sensor layer area, the tool being controlled to remove material from at least one further sensor layer area to thereby create at least one further code segment pattern. A further surface may be the opposed surface on a disc-formed carrier or the circumferential edge surface.

As used herein, the term “drug” is meant to encompass any flowable medicine formulation capable of being passed through a delivery means such as a cannula or hollow needle in a controlled manner, such as a liquid, solution, gel or fine suspension, and containing one or more drug agents. The drug may be a single drug compound or a premixed or co-formulated multiple drug compounds drug agent from a single reservoir. Representative drugs include pharmaceuticals such as peptides (e.g. insulins, insulin containing drugs, GLP-1 containing drugs as well as derivatives thereof), proteins, and hormones, biologically derived or active agents, hormonal and gene based agents, nutritional formulas and other substances in both solid (dispensed) or liquid form. In the description of the exemplary embodiments reference will be made to the use of insulin and GLP-1 containing drugs, this including analogues thereof as well as combinations with one or more other drugs.

### BRIEF DESCRIPTION OF THE DRAWINGS

In the following embodiments of the invention will be described with reference to the drawings, wherein

FIGS. 1 and 2 show a front-loaded drug delivery device with respectively without a drug cartridge mounted,

FIG. 3 shows in an exploded view a drug delivery device subassembly comprising a logging module,

FIG. 4 shows an exploded view of the logging module of FIG. 3,

FIGS. 5 and 6 show first respectively second rotary sensor parts of the module of FIG. 3,

FIG. 7A shows a sensor member in an initial state, and

FIG. 7B shows the sensor member of FIG. 7A in a machined final state.

In the figures like structures are mainly identified by like reference numerals.

### DESCRIPTION OF EXEMPLARY EMBODIMENTS

When in the following terms such as “upper” and “lower”, “right” and “left”, “horizontal” and “vertical” or similar relative expressions are used, these only refer to the appended figures and not necessarily to an actual situation of use. The shown figures are schematic representations for which reason the configuration of the different structures as well as their relative dimensions are intended to serve illustrative purposes only. When the term member or element is used for a given component it generally indicates that in the described embodiment the component is a unitary component, however, the same member or element may alternatively comprise a number of sub-components just as two or more of the described components could be provided as unitary components, e.g. manufactured as a single injection moulded part. When it is defined that members are mounted axially free to each other it generally indicates that they can be moved relative to each other, typically between defined stop positions whereas when it is defined that members are mounted rotationally free to each other it generally indicates that they can be rotated relative to each other either freely or between defined stop positions. The terms “assembly” and “subassembly” do not imply that the described components necessary can be assembled to provide a unitary or functional assembly or subassembly during a given assembly procedure but is merely used to describe components grouped together as being functionally more closely related.

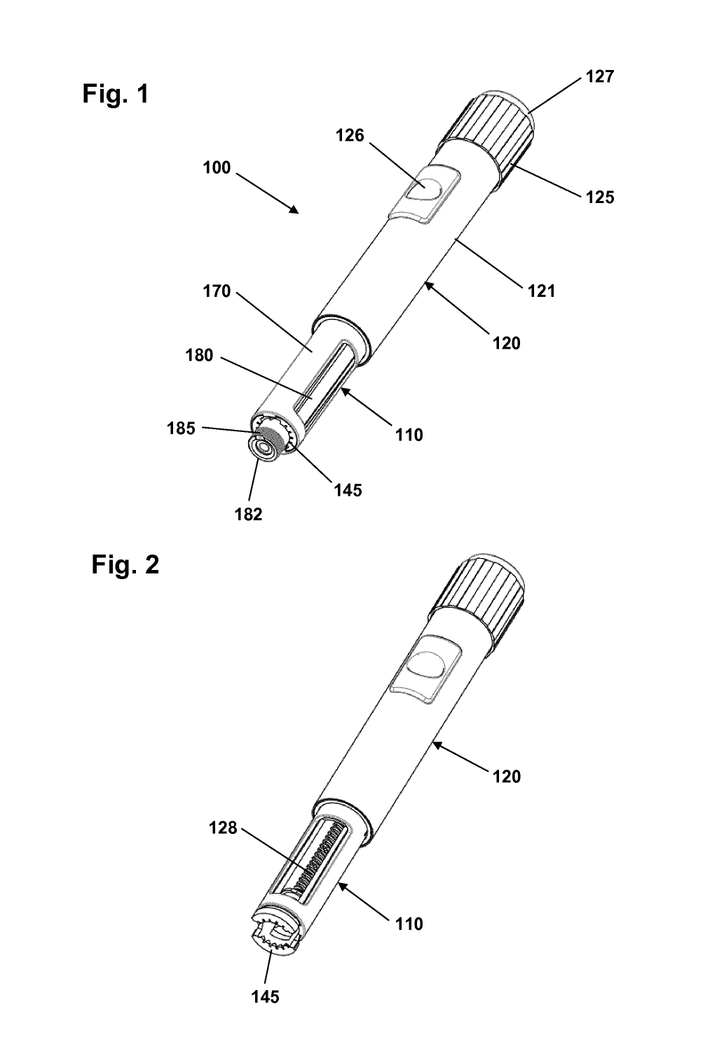

Referring to FIG. 1 a pen-formed drug delivery device 100 will be described. The device represents a “generic” drug delivery device providing an example of a device in combination with which embodiments of the present invention is intended to be used, such a device comprising a rotational member adapted to rotate corresponding to a set and/or expelled dose of drug.

More specifically, the pen device comprises a cap part (not shown) and a main part having a proximal body or drive assembly portion 120 with a housing 121 in which a drug expelling mechanism is arranged or integrated, and a distal cartridge holder portion in which a drug-filled transparent cartridge 180 with a distal needle-penetrable septum can be arranged and retained in place by a cartridge holder 110 attached to the proximal portion, the cartridge holder having openings allowing a portion of the cartridge to be inspected. The cartridge may for example contain an insulin, GLP-1 or growth hormone formulation. The device is designed to be loaded by the user with a new cartridge through a distal receiving opening in the cartridge holder, the cartridge being provided with a piston driven by a piston rod 128 forming part of the expelling mechanism. A proximal-most rotatable dose ring member 125 serves to manually set a desired dose of drug shown in display window 126 and which can then be expelled when the release button 127 is actuated. Depending on the type of expelling mechanism embodied in the drug delivery device, the expelling mechanism may comprise a spring which is strained during dose setting and then released to drive the piston rod when the release button is actuated. Alternatively the expelling mechanism may be fully manual in which case the dose ring member and the release button moves proximally during dose setting corresponding to the set dose size, and then moved distally by the user to expel the set dose.

The cartridge is provided with distal coupling means in the form of a needle hub mount 182 having, in the shown example, an external thread 185 adapted to engage an inner thread of a corresponding hub of a needle assembly. In alternative embodiments the thread may be combined with or replaced by other connection means, e.g. a bayonet coupling.

The cartridge holder comprises a distal opening adapted to receive a cartridge. More specifically, the cartridge holder comprises an outer rotatable tube member 170 operated by the user to control movement of gripping means to thereby open and close gripping shoulders 145 configured to grip and hold a cartridge. FIG. 2 shows the device with the cartridge removed and the gripping shoulders in their un-locked “open” position in which a cartridge can be removed and a new inserted.

As appears, FIG. 1 shows a drug delivery device of the front-loaded type in which a cartridge is inserted through a distal opening in the cartridge holder which in non-removable attached to the main part of the device, however, the drug delivery device may alternatively comprise a cartridge holder adapted to be removed from the device main portion and in which a cartridge is received and removed through the proximal opening.

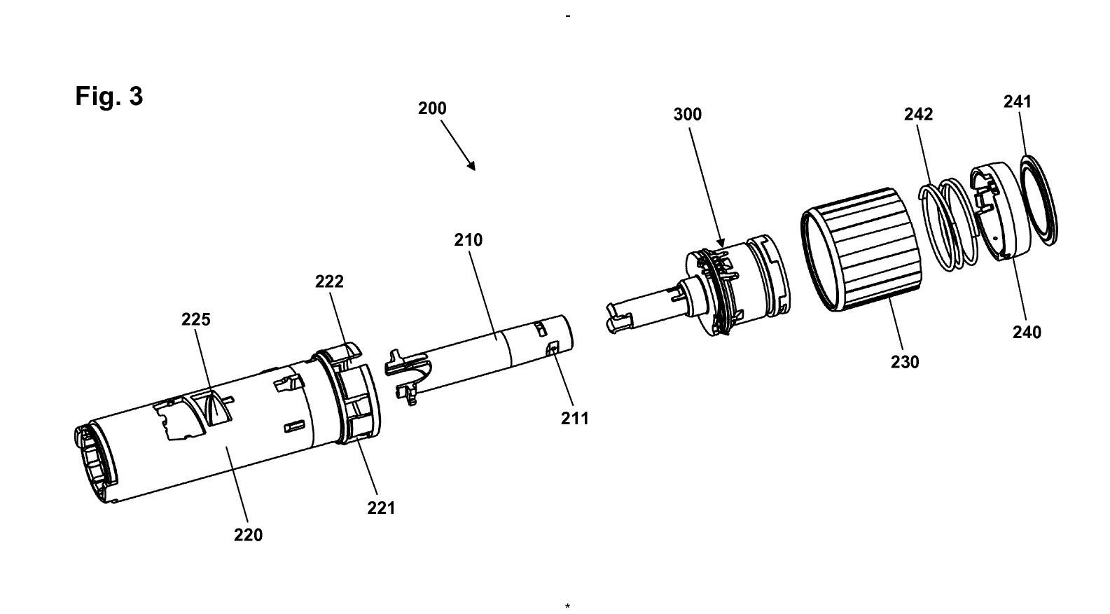

With reference to FIG. 3 a subassembly 200 for a drug delivery device will be described, the subassembly comprising a logging module in combination with parts of the drug delivery device being directly functionally related to the incorporation and operation of logging unit. More specifically, the subassembly comprises an electronically controlled logging module 300, an inner tube member 210, a generally cylindrical inner housing member 220, a dial ring member 230 and a button assembly comprising a button ring 240, a button window 241 and a button spring 242. The inner housing member is configured to be arranged inside an outer housing member providing the exterior of the drug delivery device.

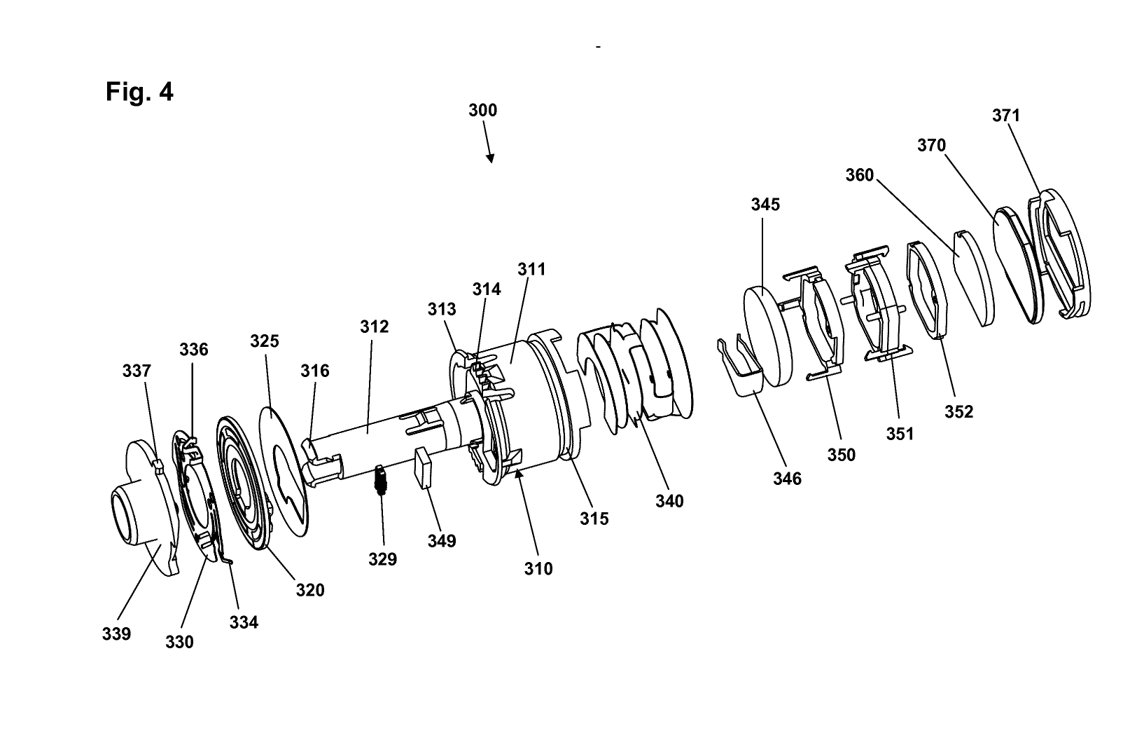

The different components of the logging module 300 are shown in FIG. 4. More specifically, the logging module comprises a housing member 310 having a barrel-shaped proximal main portion 311 with a distally extending tube portion 312, a mounting foil member 313, a disc-formed first rotary sensor part 320 onto which a first connector 329 is to be mounted, a disc-formed second rotary sensor part 330, a rotary sensor holder 339 with a lateral projection 337, a flexible PCB 340 folded in a multi-layered stack and onto which a second connector 349 is to be mounted, a battery 345 and battery clip 346, a number of mounting rings 350, 351, 352, an antenna 360, an LCD 370 and an LCD frame 371. On the PCB electronic circuitry components are mounted, e.g. micro-controller, display driver, memory and wireless communication means. As will be described below in greater detail the first rotary sensor part 320 comprises a plurality of arc-formed discreet contact areas, and the second rotary sensor part 330 comprises a plurality of flexible contact arms of which the outer ones provide an axial switch having a laterally extending projection 334.

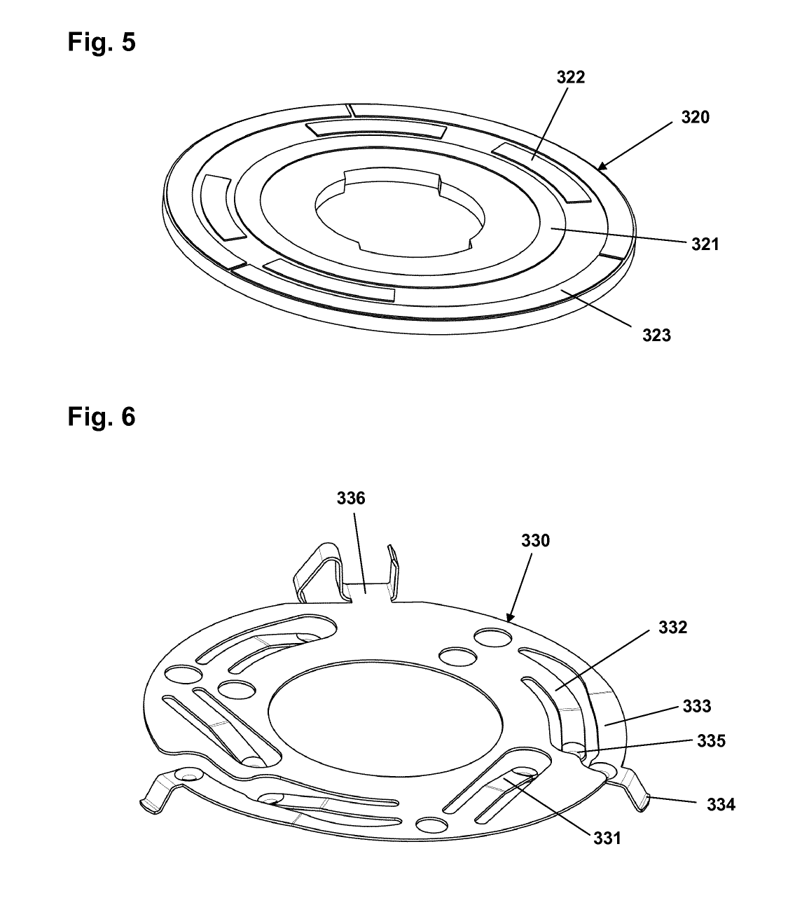

FIG. 5 shows the first rotary sensor part 320 comprising a ring-formed disc formed from circuit board material and on which a number of contact areas (or segments) has been plated on forming three concentric rings, an inner, an intermediate and an outer ring. In shown embodiment the inner ring is a single contact area 321 used as ground (i.e. reference), the intermediate ring comprises four discrete arch-formed position contact segments 322 arranged with a certain circumferential distance there between, and the outer ring comprises three discrete arch-formed switch contact segments 323 arranged with only a small circumferential gap there between, the segments being individually connected to a given contact terminal of the multi-terminal connector 329 mounted on the rear (proximal) face of the disc. If a given segment is not connected to a terminal it can be considered a passive segment.

The second rotary sensor part 330 shown in FIG. 6 is in the form of a metallic disc comprising a number of flexible arc-formed contact arms protruding proximally, the distal end of each contact arm comprising a dome-formed contact point 335 (facing downwards in the figure) adapted to create a galvanic connection with a given contact area. The contact arms are arranged corresponding to the three concentric rings of the first rotary sensor part. More specifically, the second rotary sensor part comprises two inner contact arms 331, three intermediate contact arms 332 and two outer contact arms 333.

In this way a given pair of contact arms provides a combined contact structure adapted to create electric contact between two contact segments. In the shown embodiment the two inner ground contact arms 331 are provided to be in contact with the single ground contact area 321 of the inner concentric ring, the three position contacts arms 332 are provided to be in contact with the four position contact segments 322 of the intermediate concentric ring, and the two outer switch contact arms 333 are provided to be in contact with the three switch contact segments 323 of the outer concentric ring, the outer switch contact arms carrying a laterally extending projection 334. Indeed, for the intermediate and outer contact arms the rotational position between the two sensor parts will determine which contact segment is engaged with a given contact arm.

In the shown embodiment the gaps between two neighbouring outer contact segments are dimensioned such that the dome-formed contact point will be in contact with both segments as it moves from one segment to the next, this being explained in greater detail below. The second rotary sensor part further comprises a gripping part 336 adapted to engage the projection 337 on the rotary sensor holder 339 to prevent rotational movement there between.

In the shown embodiment the intermediate arms and contact segments provide the rotary sensor contacts whereas the outer arms and contact segments provide an axial switch as will be described in greater detail below.

When manufacturing a rotary sensor part of the type shown in FIG. 5, i.e. comprising a surface having a track of code segments arranged in a pattern, the same surface having means of rotational alignment towards an external rotational reference, such that the code pattern is aligned towards that external rotational reference, it is critical for the precision and accuracy of the measuring system that the tolerances of the sensor parts are as small as possible. The challenge is to maintain a low manufacturing cost while keeping tolerances small. An example of a standard manufacturing process of such a sensor part is by manufacturing a printed circuit board (PCB) with one process for creating the code segment pattern (e.g. PCB pattern imaging, etching, stripping), and another process for creating the rotational alignment (e.g. PCB outline de-panelling by cutting/drilling the PCB material). These two different process steps contribute to the tolerance stack-up between code pattern and alignment structure.

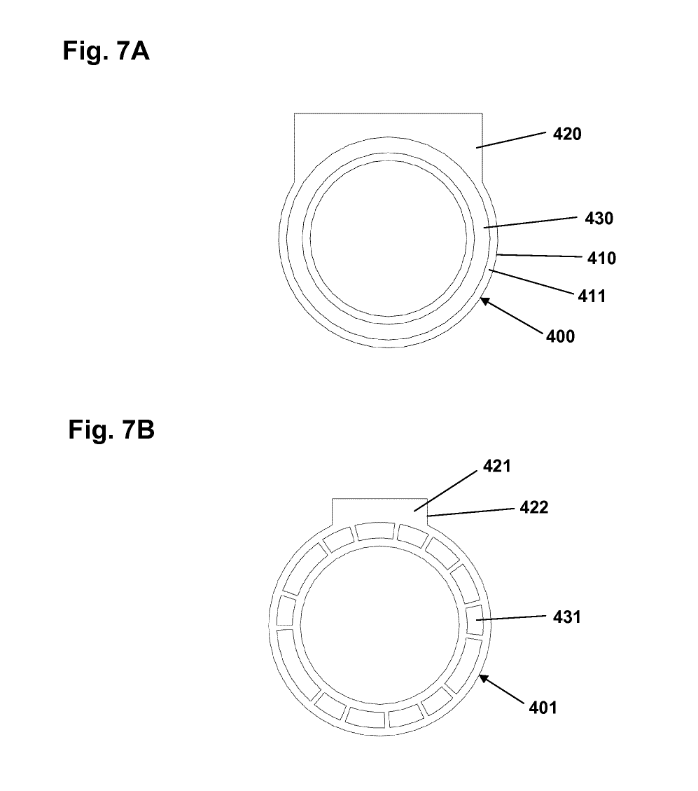

Turning to FIGS. 7A and 7B a method of manufacturing a rotary sensor part of the type shown in FIG. 5 will be described, the method providing reduced tolerance stack-up between the code pattern and the alignment structure.

FIG. 7A shows a sensor member 400 in an initial state, the sensor member comprising a ring-formed carrier 410 having an upper main surface 411 and an alignment portion 420. The carrier may be formed from traditional circuit board material. A ring-formed conducting sensor layer area 430 is formed on a portion of the carrier main surface, e.g. by plating. To create the code segment pattern and the rotational alignment structure the sensor member is arranged in a fixed position, e.g. by mechanical clamping, relative to a material-removing tool adapted to remove material from the carrier alignment area as well as the sensor layer. Subsequently the material-removing tool is controlled to (i) remove material from the carrier alignment portion to thereby create an alignment structure, and (ii) remove material from the sensor layer area to thereby create a code segment pattern, each code segment thereby being aligned with the alignment structure. The material-removing tool may be an ablation laser operated at a first intensity and/or speed to cut the alignment area corresponding to its final form, and operated at a second intensity and/or speed to remove only the relatively thin layer of the conducting sensor layer corresponding to the desired pattern.

By combining the two process activities in one step, the tolerances inherent when aligning the surface between the two process activities are eliminated, and only the inherent tolerances in the single process of creating the pattern and alignment structure are present, e.g. the accuracy/precision of a laser ablation system.

FIG. 7B shows a final sensor member 401 formed from the initial sensor member of FIG. 7A.

The initial alignment portion has been cut to a final alignment portion 421 having an alignment structure in the form of well-defined edges 422, and with the ring-formed conducting sensor layer cut into a number of individual segments 431. If desired the specific sensor member of FIG. 5 could be formed using the described method.

In alternative embodiments the carrier member may be provided with conducting sensor layers on additional surfaces, e.g. the opposed surface or the edge surface. Indeed, if the outer circumferential edge should be used for sensor purposes the alignment structure should be arranged at an alternative location, e.g. as shown in FIG. 5. Also, more than one alignment structure may be formed. Instead of a rotational sensor a linear sensor member may be formed.

In the above description of exemplary embodiments, the different structures and means providing the described functionality for the different components have been described to a degree to which the concept of the present invention will be apparent to the skilled reader. The detailed construction and specification for the different components are considered the object of a normal design procedure performed by the skilled person along the lines set out in the present specification.

1. A method of manufacturing a sensor member, comprising the steps of:
providing a sensor member in an initial state, the sensor member comprising:
a carrier having a surface and an alignment portion, and
at least one conducting sensor layer area formed on a portion of the carrier surface,
providing a material-removing tool adapted to remove material from the carrier alignment portion and the sensor layer,
arranging the sensor member in a fixed position relative to the tool, and
control the tool to:
remove material from the carrier alignment portion to thereby create an alignment structure, and
remove material from at least one sensor layer area to thereby create a code segment pattern, each code segment thereby being aligned with the alignment structure.

2. A method as in claim 1, wherein the created alignment structure is an edge portion of the carrier.

3. A method as in claim 1, wherein the carrier is in the form of a planar board.

4. A method as in claim 1, wherein the at least one conducting sensor layer area is formed on the carrier surface by a plating process.

5. A method as in claim 1, wherein the tool is an ablation laser.

6. A method as in claim 1, wherein at least one conducting sensor layer area is ring-formed, the created code segments being in the form of a plurality of annular segments each spanning a given number of degrees.

7. A method as in claim 1, wherein a number of alignment structures are created.

8. A method as in claim 1, wherein the provided carrier has one or more additional surfaces each comprising at least one conducting sensor layer area.

9. A method as in claim 8, wherein the tool is controlled to remove material from at least one further sensor layer area to thereby create at least one further code segment pattern.
